(19) United States
(12) Patent Application Publication
(10) Pub. No.: US 2017/0340792 A1
(43) Pub. Date: Nov. 30, 2017

(54) A PORTABLE REGENERATIVE HAEMODIALYSIS SYSTEM AND A METHOD THEREOF

(71) Applicant: RENALYX HEALTH SYSTEMS PVT LTD, Bangalore, Karnataka (IN)

(72) Inventors: Vishnu SHYAM, Bangalore (IN); Mahabaleswara Ram BHATT, Bangalore (IN); Kishin GANDHI, Bangalore (IN); Shyam Vasudev RAO, Bangalore (IN)

(73) Assignee: RENALYX HEALTH SYSTEMS PVT LTD, Bangalore, Karnataka (IN)

(21) Appl. No.: 15/022,837

(22) PCT Filed: Sep. 9, 2015
(86) PCT No.: PCT/IB2015/056895

§ 371 Date: Mar. 17, 2016

(30) Foreign Application Priority Data
Feb. 19, 2015 (IN) 794/CHE/2015

(51) Int. Cl. A61M 1/16; H01M 16/00

(52) U.S. Cl. CPC A61M 1/16; H01M 16/006; A61M 2205/8293; A61M 2205/3324; A61M 2205/8206; A61M 2205/3368; A61M 2205/3331; H01M 2250/30; A61M 2205/8268

(57) ABSTRACT

The present disclosure provides a portable haemodialysis system, comprising at least one haemodialyser configured to purify a biological sample. At least one fuel cell is fluidly connected to the at least one haemodialyser, the at least one fuel cell is adapted to receive oxygen and hydrogen from at least one oxygen and hydrogen storage units to generate energy and water. At least one energy reservoir is connected to the at least one fuel cell which is configured to store the energy generated in the at least one fuel cell. The water generated in the at least one fuel cell is supplied to the at least one haemodialyser for purification of the biological sample, and the energy stored in the at least one energy reservoir is used to power the haemodialyser during operation.

### TECHNICAL FIELD

The present disclosure generally relates to bio-medical devices. Particularly but not exclusively, the present disclosure relates to a haemodialysis system for purifying biological samples. Further, embodiments of the disclosure disclose a portable regenerative haemodialysis system and a method of operating the same.

### BACKGROUND OF THE DISCLOSURE

Haemodialysis is the most commonly employed method or a treatment to treat advanced and permanent kidney disorders. In recent years, more compact, simple and cost-effective haemodialysis systems have been developed to treat a disorder which is most commonly associated with kidneys which is the Chronic Kidney Disease (CKD).

Healthy kidneys in living beings purify blood by removing unwanted fluids, minerals and wastes from it. The kidneys also generate hormones to keep bones strong and blood healthy, and help to control acid/base balance in the body. When kidneys fail in the living body, harmful wastes and fluids build up in the body which may progressively lead to serious complications, ultimately leading to death. Chronic Kidney Disease (CKD) is one such disease which is asymptomatic i.e. cannot be detected till an advanced stage. CKD is commonly found in diabetics and people with high blood pressure, leading to many other disorders such as anaemia, weak bones, low nutritional health and nerve damage. With the advancement in science and technology, some bio-medical devices such as haemodialysis systems or artificial kidneys are developed, and are used extensively to function as substitutes for kidneys. These systems are used for blood purification when kidneys no longer perform adequately or when they have been removed.

The conventional haemodialysis systems are built by incorporating a dialysis membrane in a housing, dividing the housing into two compartments. The first compartment stores blood sample collected from the subject and the second compartment collects dialysate which is used for purification of blood. The dialysis membrane is placed in between the two compartments and is shaped in such a way that mass transfer occurs between the blood and dialysate through it. The dialysis membrane is a partially permeable membrane which allows only the particles and/or solvent of predetermined size to diffuse through it.

The principle of haemodialysis involves two distinct mechanisms diffusion and hydrostatic pressure gradient. The diffusion is a mass transfer phenomenon and is defined as net spontaneous movement of particles from a region of higher concentration to a region of lower concentration i.e. spontaneous movement of particles due to concentration gradient. The particles may be atoms, ions or molecules. On the other hand, hydrostatic pressure gradient is defined as variation in pressure difference along a predetermined distance. Either a concentration gradient (diffusion) or a pressure gradient or both may be employed to carry out haemodialysis process across the dialysis membrane.

FIG. 1 is a schematic diagram illustrating a conventional haemodialysis system. The system comprises of a dialysis machine where corporeal fluids such as but not limiting to blood is purified by another fluid such as water. The purification is achieved through diffusion and by varying hydrostatic pressure gradient. The dialysis machine is provided with associated inputs ingredients such as water and electricity. A reservoir is used to store water with high degree of purity, which is pumped into the water compartment of the dialysis machine. The water which drains out of the dialysis machine after removing unwanted wastes from the blood is collected in a drain chamber. In most of the conventional dialysis systems, the pure water reservoir is supplied with fresh water having high degree of purity, and there is no provision to recycle and/or regenerate pure water from it. In few other dialysis systems, drain water is purified by subjecting it to processes like distillation followed by condensation as shown in FIG. 2.

Normally the dialysis machine comprises of a blood port through which the blood to be purified is passed. The wall of the blood port is made of a semi-permeable membrane for diffusion of wastes and unwanted fluids. The blood port is surrounded by a shell/cylinder through which the dialysate (a solvent with mineral ions dissolved in it) is passed in opposite direction to the flow of blood i.e. in a counter-current fashion. During the counter-current flow, solid wastes like urea, salts of potassium and sodium, and other solutes diffuse into the dialysate due to concentration gradient. Ultrafiltration (removal of unwanted fluids) is achieved by varying hydrostatic pressure gradient across the membrane.

There are many drawbacks and complications associated with conventional haemodialysis systems, which includes requirement of ultra-high quality' water, requirement of continuous supply of electricity, highly skilled personnel for handling and cleaning the dialysis apparatus, constant regulation of parameters such as blood and dialysate flow pressures, and so on. Of these, supplying ultra-pure water to produce the dialysate by dissolving mineral ions in it is of major concerns. An extensive water purification system is absolutely critical for haemodialysis. Since dialysis patients are exposed to vast quantities of water which is mixed with dialysate concentrate to form the dialysate, there are chances that even traces of mineral contaminants or bacterial matter can filter into the patient's blood. Since the damaged kidneys cannot perform their intended function of removing impurities, ions introduced into the bloodstream via water in the dialysis machine can build up to hazardous levels, causing numerous undesirable symptoms, or may even prove fatal.

To overcome these limitations associated with water supply and handling, the water used as dialysate is subjected to various stages of purification, which is summarized as follows:

Initially it is subjected to filtration which removes suspended and colloidal impurities by entrainment. This is followed by temperature adjustment where temperature of water is brought to a predetermined value suitable for supplying it into the dialysis system. It is then subjected to pH adjustments to match with the pH value of blood by adding acid/base. This is followed by softening which involves removal of salts causing hardness by one or more chemical treatments. Organic compounds are removed by mechanical methods such as adsorption. Lastly, water is subjected to processes like Reverse Osmosis (R.O) for obtaining high purity.

The above described stages involved in obtaining water with high degree of purity are tedious, time-consuming, involve complicated treating procedures, require sophisticated conditions, and are inherently expensive. In addition, highly skilled personnel are required to perform each of these water treatment operations, which make the process less feasible in places where field of medicine is not so advanced and in places where availability of resources is minimal. Also, the haemodialysis equipment cannot be installed in remote locations where availability of water and electricity is less, since water and electricity are the major input ingredients required to operate the haemodialyser, and consequently, to perform the haemodialysis treatment. These factors and limitations make the conventional haemodialysis systems inaccessible to the people residing in remote locations and in locations where medical science is not well established.

Further, it is less possible or rather impossible to obtain water with very high degree of purity (ultra-pure water) by following the above described sequence of purification steps, owing to manual intervention and lack of availability of favourable conditions. Also, conventional haemodialysis systems are installed/set up in medical practitioners' place such as in clinical laboratories or in hospitals, where patients with CKD or other kidney related disorders are brought to the clinical laboratories or to hospitals to undergo dialysis treatment on a periodic basis, depending on the severity. However, in cases of emergencies/casualties where patient is unable to come to haemodialysis system to undergo the dialysis treatment, this may not be viable. In such scenarios, conventional dialysis systems cannot be relied upon.

In light of foregoing discussion, it is necessary to develop portable regenerative haemodialysis system to overcome one or more limitations stated above.

### SUMMARY OF THE DISCLOSURE

The one or more limitations of conventional haemodialysis systems as described in the prior art are overcome and additional advantages are provided through the system as claimed in the present disclosure. Additional features and advantages are realized through the technicalities of the present disclosure. Other embodiments and aspects of the disclosure are described in detail herein and are considered to be a part of the claimed disclosure.

In one non-limiting embodiment of the present disclosure, there is provided a portable haemodialysis system. The system comprises at least one haemodialyser configured to purify a biological sample. At least one fuel cell is fluidly connected to the at least one haemodialyser, the at least one fuel cell is adapted to receive oxygen and hydrogen from at least one oxygen and hydrogen storage units to generate energy and water. At least one energy reservoir is connected to the at least one fuel cell, the at least one energy reservoir is configured to store the energy generated in the at least one fuel cell. The water generated in the at least one fuel cell is supplied to the at least one haemodialyser for purification of the biological sample, and the energy stored in the at least one energy reservoir is used to power the haemodialyser during operation.

In an embodiment of the present disclosure, the biological sample is a corporeal fluid.

In an embodiment of present disclosure, the at least one fuel cell is a Hydrogen-Oxygen fuel cell and the at least one energy reservoir is a battery.

In an embodiment of the present disclosure, at least one energy reservoir is connected to at least one alternate energy source.

In an embodiment of the present disclosure, at least one water reservoir is fluidly connected to water outlet of the haemodialyser.

In an embodiment of the present disclosure, at least one water purification unit is fluidly connected to the water reservoir and the haemodialyser, the at least one water purification unit is configured to purify the water used for purification of the biological sample.

In an embodiment of the present disclosure, at least one water splitting unit is fluidly connected to at least one of the at least one water purification unit and the at least one water reservoir. The at least one water splitting unit is configured to regenerate gaseous hydrogen and oxygen by decomposing the pure water. Further, the at least one water splitting unit comprises outlet ports, the outlet ports are fluidly connected to the oxygen and hydrogen storage units for supplying gaseous oxygen and hydrogen.

In an embodiment of the present disclosure, at least one sensor is interfaced with a control unit for detecting and indicating the energy level in the at least one energy reservoir.

In an embodiment of the present disclosure, at least one flow regulation unit is provisioned in flow passages between the oxygen and hydrogen storage units and the fuel cell. The at least one flow regulation unit is interfaced with the control unit for regulating the flow of hydrogen and oxygen to the at least one fuel cell.

In another non-limiting embodiment of the present disclosure, there is provided a regenerative portable haemodialysis system. The system comprises at least one haemodialyser configured to purify a biological sample. At least one fuel cell is fluidly connected to the at least one haemodialyser, the at least one fuel cell is adapted to receive oxygen and hydrogen from at least one oxygen and hydrogen storage units and is configured to generate energy and water. At least one energy reservoir is connected to the at least one fuel cell, the at least one energy reservoir is configured to store energy generated in the at least one fuel cell. The water generated in the at least one fuel cell is supplied to the at least one haemodialyser for purification of the biological sample, and the energy stored in the at least one energy reservoir is used to power the haemodialyser during operation. The system further comprises a regenerative system for regenerating water and energy used for haemodialysis. The regenerative system comprising at least one water purification unit fluidly connected to water outlet of the haemodialyser. The at least one water purification unit is configured to purify the water used for purification of the biological sample. At least one water splitting unit is fluidly connected to the at least one water purification unit, the at least one water splitting unit is configured to regenerate gaseous hydrogen and oxygen by decomposing the water, and supply the gaseous oxygen and hydrogen to the at least one oxygen and hydrogen storage units.

In another non-limiting embodiment of the present disclosure, there is provided a method for operating a regenerative portable haemodialysis system. The method comprising acts of firstly generating water and energy using at least one fuel cell by supplying oxygen and hydrogen, the at least one fuel cell is configured to receive oxygen and hydrogen from at least one oxygen and hydrogen storage tanks. Then, storing the energy generated by the at least one fuel cell in at least one energy reservoir and supplying the water generated by the at least one fuel cell to the at least one haemodialyser for purification of the biological sample. Further, the method comprises act of supplying the energy stored in the at least one energy reservoir to power the haemodialyser during operation. The method also comprises act of regenerating water and energy used for haemodialysis by a regenerative system. The regeneration comprising steps of purifying the water in at least one water purification unit, the at least one water purification unit receives water from water outlet of the haemodialyser. Then, regenerating gaseous oxygen and hydrogen by decomposing the water in at least one water splitting unit, the at least one water splitting unit receives water from the at least one water purification unit. This is followed by supplying regenerated oxygen and hydrogen to the at least one oxygen and hydrogen storage units. The at least one oxygen and hydrogen storage units are configured to receive oxygen and hydrogen from the at least one water splitting unit.

In an embodiment of the present disclosure, the method comprises act of supplying energy from at least one alternate energy source of the at least one energy reservoir.

In an embodiment of the present disclosure, the method comprises act of regulating flow of oxygen and hydrogen from the at least one oxygen and hydrogen storage units to the at least one fuel cell by at least one flow regulation unit.

It is to be understood that the aspects and embodiments of the disclosure described above may be used in any combination with each other. Several of the aspects and embodiments may be combined together to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The novel features and characteristics of the disclosure are set forth in the appended description. The disclosure itself, however, as well as a preferred mode of use, further objectives and advantages thereof, will be best understood by reference to the following detailed description of an illustrative embodiment when read in conjunction with the accompanying figures. One or more embodiments are now described, by way of example only, with reference to the accompanying figures wherein like reference numerals represent like elements and in which:

FIG. 1 illustrates the schematic representation of a conventional haemodialysis system along with various units for supplying sustainable ingredients.

FIG. 2 illustrates the schematic representation of water purification system interfaced with conventional haemodialysis system of FIG. 1.

FIG. 3 illustrates the schematic representation of haemodialysis system with arrangements to supply sustainable ingredients, according to an embodiment of the present disclosure.

FIG. 4 illustrates the schematic representation of haemodialysis system of FIG. 3 along with water splitting unit, according to some embodiment of the present disclosure.

FIG. 5 illustrates the schematic representation of haemodialysis system of FIG. 4 along with the water purification unit, according to some embodiment of the present disclosure.

FIG. 6 illustrates an exemplary embodiment of the haemodialysis system of the present disclosure comprising one or more batteries as energy reservoirs, distillation and condensation unit as water purification unit and at least one solar cell module as alternate energy source.

FIG. 7a illustrates the schematic representation of control system associated with the haemodialysis system for balanced generation of dialysis ingredients, according to some embodiment of the present disclosure.

FIG. 7b illustrates exemplary truth table depicting a logic of control system for balanced generation of dialysis ingredients, according to some embodiment of the present disclosure.

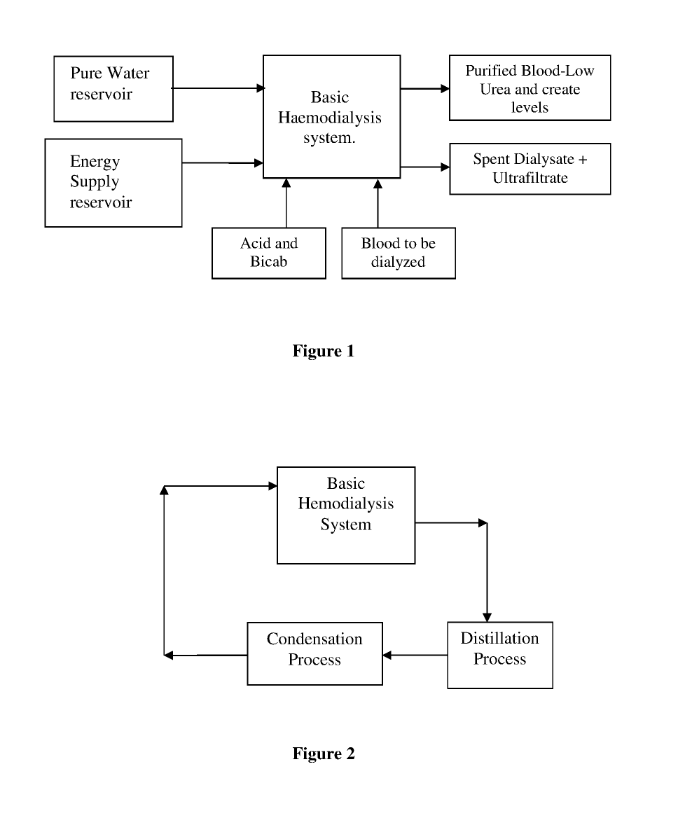

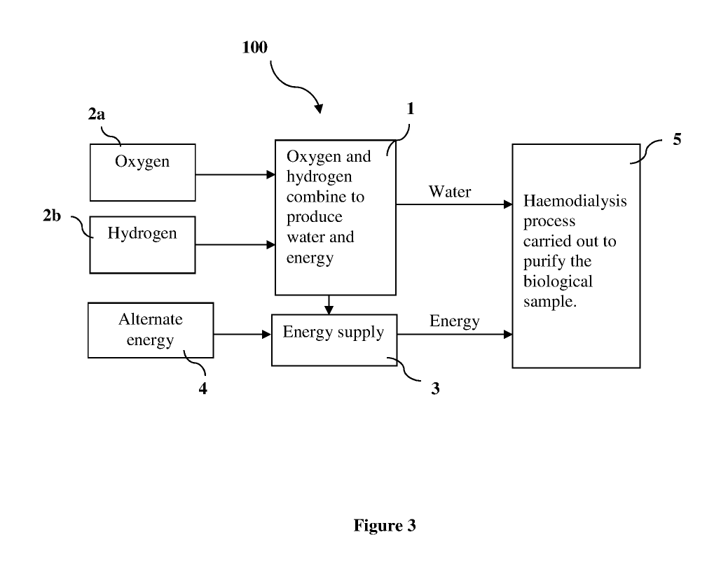

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

To overcome one or more limitations stated in the background, the present disclosure provides a regenerative, portable hemodialysis system with an arrangement for balanced, self-sustained supply of ingredients (mainly water and energy) to it. The haemodialysis system comprises of at least one fuel cell to generate required quantities of pure water and energy, which are principal input ingredients to the haemodialysis system. The generated water is supplied to the haemodialysis machine to carry out the process of purification of biological sample, such as but not limiting to blood. In addition to water, energy is generated in the fuel cell which is used to power the haemodialysis machine and other components in the system. The energy produced in the fuel cell is supplied to at least one energy reservoir. The energy reservoir is configured to store the energy and deliver it to the haemodialysis machine during operation, and also to other auxiliary components constituting the haemodialysis system. The energy reservoir is also separately connected to an alternate energy source such as but not limiting to one or more solar cell modules, so as to make the energy reservoir two-way powered. In other words, the energy reservoir is configured such that it is powered by alternate energy source during the availability of alternate energy, and alternatively by the energy generated in fuel cell by virtue of electro-chemical reaction between gaseous components supplied to the fuel cell. The gaseous components, being oxygen and hydrogen, are required to generate water and electricity in the fuel cell. These gaseous components are stored in at least one oxygen storage unit and at least one hydrogen storage unit. The oxygen and hydrogen storage units are fluidly connected to the fuel cell to supply oxygen and hydrogen to the fuel cell at required flow rates and pressures.

The haemodialysis system further comprises of a regenerative system which is adapted to regenerate input ingredients. The regenerative system comprises at least one water reservoir fluidly connected to water outlet of the haemodialysis machine. The water reservoir receives and stores waste water (interchangeably referred to as “drain water” throughout the specification) flowing out of the haemodialysis machine. The outlet port of the water reservoir is fluidly connected to at least one water purification unit. The waste water stored in water reservoir is purified by the water purification unit to make the water suitable for regeneration. Then, the regeneration of water to obtain oxygen and hydrogen is achieved through at least one water splitting unit which is in fluid communication with the water purification unit. Alternatively, the water splitting unit may be disposed directly in fluid communication with the water reservoir so that water stored in the water reservoir is directly routed into the water splitting unit for regenerating oxygen and hydrogen. In an embodiment of the disclosure, the water splitting unit chemically decomposes pure water into gaseous oxygen and hydrogen. The gaseous oxygen and hydrogen so regenerated are supplied to oxygen and hydrogen storage units for supplying it to the fuel cell, and consequently to generate water and energy in the fuel cell to repeat the haemodialysis process. In this way, volumes of oxygen and hydrogen are replenished (or restored) in the oxygen and hydrogen storage units, so that continuous availability of the gases for circulation into the fuel cell is ensured. The energy required to operate water purification unit and the water splitting unit is supplied by the at least one energy reservoir. The pressure and flow rates of gaseous hydrogen and oxygen flowing from the respective storage units into the fuel cell are regulated by one or more flow regulation units provided in fluid flow lines, including but not limiting to pressure and flow control valves.

The haemodialysis system still further comprises of one or more sensors for detecting energy levels in the energy reservoir. The one or more sensors are interfaced with a control unit, and the control unit receives signals corresponding to the energy level. The control unit is also interfaced with the haemodialysis system and the regenerative system, and is configured to control various processes and process parameters involved in the operation of haemodialysis system. In an embodiment of the disclosure, the control unit regulates the flow of gaseous oxygen and hydrogen from storage units into the fuel cell, and also from water splitting unit into the storage units. In addition, the control unit receives input signals from sensors to maintain energy level in the energy reservoir, which makes the fuel cell operate uninterruptedly during the haemodialysis process. The control unit is preloaded with appropriate controller logic to control flow of ingredients (mainly oxygen and hydrogen) between components, and also to control various other process parameters, which will be described in detail in forthcoming paragraphs of detailed description.

Use of terms such as “comprises”, “comprising”, or any other variations thereof in the description are intended to cover a non-exclusive inclusion, such that a setup system, device, assembly or method that comprises a list of components or steps does not include only those components or steps but may include other components or steps not expressly listed or inherent to such setup or device or method. In other words, one or more elements in a mechanism proceeded by “comprising . . . a” does not, without more constraints, preclude the existence of other elements or additional elements in the mechanism.

Reference will now be made to a portable regenerative haemodialysis system, and is explained with the help of figures. The figures are for the purpose of illustration only and should not be construed as limitations on the system. Wherever possible, referral numerals will be used to refer to the same or like parts.

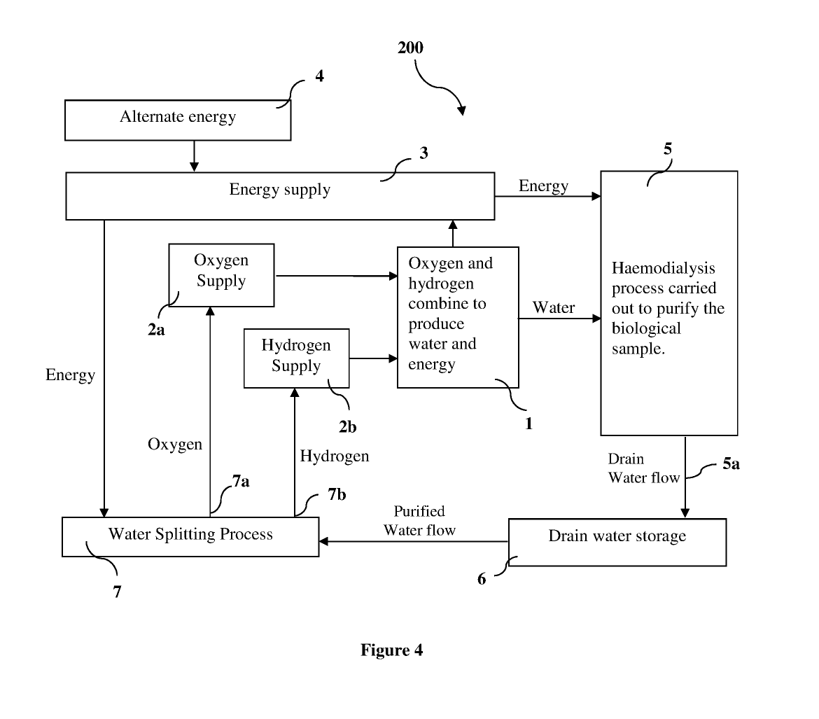

FIG. 3 is an exemplary embodiment of the present disclosure which illustrates schematic block diagram of a haemodialysis system (100), along with various units for supplying sustainable ingredients. The haemodialysis system (100) comprises of a haemodialyser (5) (interchangeably referred to as haemodialysis machine throughout the detailed description). The haemodialyser (5) comprises of two chambers—one to store at least one corporeal fluid such as but not limiting to human blood, and other chamber to store water with high degree of purity. The corporeal fluids are pumped into the haemodialyser (5) from patient's body using a circulation pump [not shown] at required pressure and flow rate. Similarly, water is pumped into the haemodialysis machine (5) using a water pump [not shown]. The corporeal fluid and water are circulated inside the haemodialyser (5) to carry out the purification process. The direction of flow of corporeal fluid and water in the respective compartments of haemodialysis machine (5) includes but not limiting to counter-current flow. Further, pure water is added with minerals so that it is made suitable for removing unwanted solid and fluids from the corporeal fluid through diffusion and hydrostatic pressure gradient. The haemodialysis machine (5) further comprises of a semi-permeable membrane (called dialyzer membrane) placed in between the fluid chamber and the water chamber. This membrane acts as a separation medium between the corporeal fluid and pure water, and allows only the particles with certain size (or lesser) to pass through them, depending on pore size. In an embodiment of the present disclosure, the semi-permeable membrane is made of a material, including but not limiting to cellulose.

The haemodialysis machine (5) requires continuous supply of input ingredients to operate. Water and energy are the chief input ingredients which are supplied to the haemodialysis machine (5). Water is intended to perform purification of a biological sample includes but not limited to corporeal fluids, for example human blood. On the other hand, energy has to be supplied to the haemodialysis machine (5) to receive the biological sample from subject to carry out purification process. Once the purification process is accomplished, the purified biological sample has to be supplied back to the subject, which again requires energy. In a similar manner, energy is required to circulate pure water into the haemodialysis machine (5), and to expel or drain waste water from the haemodialysis machine (5) after the purification process is complete. The power required for transportation of biological samples and water from and to the haemodialyser (5) is supplied by at least one energy reservoir (3). The energy reservoir (3) is configured to supply power to the haemodialyser (5) when haemodialysis treatment is being carried out, and to operate fluidic devices in the haemodialysis system (100), including but not limiting to pumps, valves and pressure control devices. The fluidic devices take part in regulating direction of flow, pressure and other flow characteristics of corporeal fluids and water within the haemodialysis system (100). In an embodiment of the present disclosure, the energy supplied and stored in the energy reservoir (3) includes, but not limited to heat energy, electrical energy and fluid power. The type of energy used depends on size, operating characteristics and availability of that particular energy source to continuously power the haemodialysis system (100). The water supplied to operate the haemodialyser (5) to perform purification process is recycled and regenerated to produce gaseous oxygen and hydrogen.

The haemodialysis system (100) in accordance with the present disclosure is configured with an integrated unit in which both water and energy are generated. In an embodiment of the disclosure, the integrated unit is a fuel cell (1) which operates the haemodialysis system (100). A fuel cell (3) is a device which converts chemical energy of a fuel into energy through a single step chemical reaction or a sequence of chemical reactions. The basic working principle of the fuel cell (1) includes process such as fuel undergoing a chemical reaction, such as oxidation, in the presence of electrodes (anode and cathode), electrolyte and optionally one or more catalysts. The chemical reaction results in the formation of electrons and positively charged Hydrogen ions which are attracted towards the respective electrodes, thereby producing energy. In addition to energy, the fuel cell (1) produces water and small quantities of by-products depending on the composition of the fuel used. In an embodiment of the present disclosure, the fuel cell (1) includes, but not limited to hydrogen-oxygen fuel cell, Proton exchange membrane fuel cell and methanol oxygen fuel cell. The energy generated by the fuel cell (1) depends on quantities of oxygen and hydrogen supplied, size of the fuel cell (1), rate of chemical reaction taking place inside the fuel cell (1) and various other process parameters. The fuel cell (1) is connected to at least one energy reservoir (3) in which the energy generated may be stored.

Further, as shown in FIG. 3, the fuel cell (1) is in fluid communication with at least one oxygen storage unit (2a) and at least one hydrogen storage unit (2b) which supply required quantities of gaseous oxygen and hydrogen to the fuel cell (1). In an embodiment of the present disclosure, the oxygen and hydrogen storage units (2a, 2b) include but not limiting to gas storage cylinders in which the gases are stored under pressure. A fluid flow line between the storage units (2a, 2b) and the fuel cell (1) are provided with pressure control devices and flow control devices [not shown] to regulate the pressure and flow rates respectively of the gases (oxygen and hydrogen). In an embodiment of the present disclosure, the pressure control devices include, but are not limited to mechanical pressure control valves equipped with one or more pressure gauges, such as but not limiting to mechanical pressure gauges, for example, manometer and barometer. In another embodiment of the present disclosure, the flow control valves, include but are not limited to mechanical and hydraulic flow control valves such as actuators.

The haemodialysis system (100) also comprises at least one energy reservoir (3) which is connected to the fuel cell (1), and is configured to store energy generated during the electrochemical reaction taking place in the fuel cell (1). In an embodiment of the present disclosure, the energy reservoir (3) includes, but not limited to electric battery which stores electric power, or a thermic device which converts thermal energy generated in the fuel cell into a convenient form of energy, or a hydraulic device which utilizes fluid power of the fluid generated as by-product in the fuel cell (1). As far as thermal energy storage and utilization is concerned, a thermic device may be used to exploit the heat energy generated in the fuel cell (1) by converting the heat either into electrical energy, or into another convenient usable form of energy which serves the purpose. In an embodiment of the present disclosure, the thermic device includes, but not limited to a thermo-electric device which converts heat into electricity. In another embodiment of the present disclosure, the thermo-electric device includes, but not limited to thermo-electric generators. As an alternative to the thermo-electric devices, phase change materials can be employed to store the thermal energy generated in the fuel cell (1) and utilize the same whenever requirement arises. The energy stored in the phase change materials (PCM) is in the form of latent heat and this energy is released whenever there is a change of phase of material which is being used.

In an exemplary embodiment of the present disclosure, an electric battery is used as energy reservoir (3) in the portable haemodialysis system (100) to store and deliver electric power to haemodialyser (5) and associated components in the haemodialysis system (100). The terminals of the battery are connected to the fuel cell (1) so that the electricity generated by the electro-chemical reaction taking place in the fuel cell (1) is stored and made available for utilization. Electrical energy stored in the battery is used to power the haemodialysis system (100), since the fuel cell (1) generates sufficient amperes of electric current which can be easily collected in one or more batteries. In addition, the supply of electricity to haemodialyser (5) and other constituents of the haemodialysis system (100) can be easily monitored and controlled. Usage of batteries for storing and delivering electricity makes the haemodialysis system (100) portable, compact and economical. In an embodiment of the present disclosure, the energy reservoir (3) is connected to at least one alternate energy source (4) includes, but not limited to non-conventional energy source. This makes the energy reservoir (3) two-way powered so that whenever alternate energy is sufficiently available, it can be utilized or stored. The availability of alternate energy reduces load on the fuel cell (1) and ensures all time availability of the energy in the energy reservoir (3). In the absence of sufficient alternate energy, rate of energy generation inside the fuel cell (1) is slightly increased either by increasing the flow rates of oxygen and hydrogen, or by increasing the rate of chemical reaction taking place in the fuel cell (1), or by any other technique which serves the purpose. In an embodiment of the disclosure, the alternate energy source (4) includes, but not limited to solar panels, power grids, and the like.

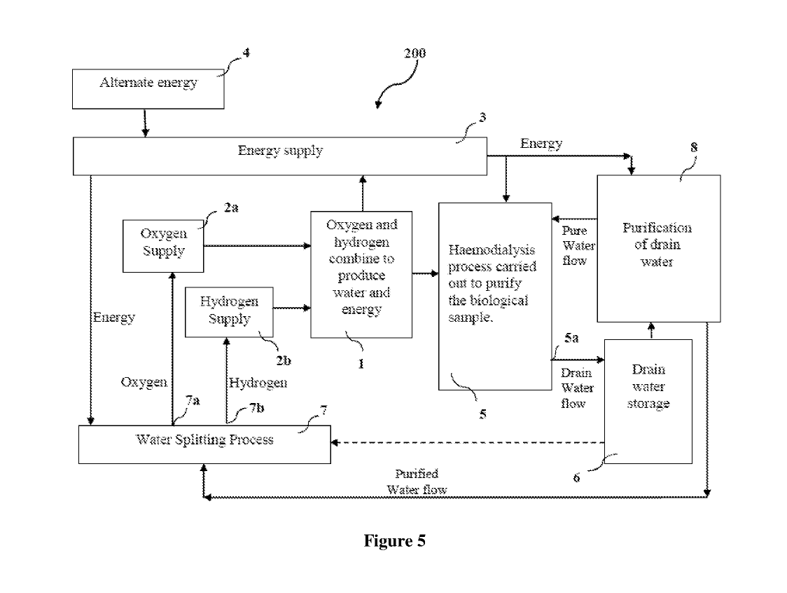

Reference is now made to FIG. 4 which is an exemplary embodiment of the present disclosure illustrating schematic diagram of regenerative haemodialysis system (200). The system (200) includes all the components as explained in above paragraphs, and additionally comprises at least one water splitting unit (7) and at least one water purification unit (8). After purification of biological sample in the presence of water, the haemodialysis system (5) has to expel waste water out of the water chamber (or compartment) for letting in pure water from the fuel cell (1) to perform next cycle of biological sample purification. The waste water so expelled is provided to at least one water reservoir (6) which collects drain water coming out of the haemodialysis machine (5). The at least one water reservoir (6) is fluidly connected to water outlet (5a) of the haemodialysis machine (5) for receiving the water. In an embodiment of the disclosure, the at least one water reservoir (6) is disposed downstream of the haemodialysis machine (5). The drain water contains miscible, immiscible and dissolved impurities in both solid and liquid phases that are to be removed from the biological sample. Removal of drain water from the haemodialysis machine (5) has to be performed in a continuous manner during each cycle of biological sample purification, which otherwise may result in contamination of fresh water flowing into the haemodialysis machine (5), as well as of the purified biological sample.

The system (200) includes a water splitting unit (7), where a part of the drain water collected in the at least one water reservoir (6) which is subjected to splitting process to generate gaseous oxygen and hydrogen. The water splitting unit (7) is provisioned in fluid communication with the water reservoir (3) and is adapted to split water to make it reusable. In an embodiment of the disclosure, the water splitting process is a chemical process where water in molecular state is chemically decomposed into gaseous hydrogen and oxygen. The decomposition of water into gaseous oxygen and hydrogen requires energy, and this energy is supplied by the at least one energy reservoir (3) connected to the water splitting unit (7). The energy is required to break the intermolecular bonds of water molecule and to produce molecular oxygen and hydrogen, which are released as gases. In an embodiment of the present disclosure, energy supplied to water splitting unit (7) to decompose water into gaseous hydrogen and oxygen includes, but not limited to electrical energy, and resulting decomposition phenomenon may be an electro-chemical phenomenon includes, but not limited to electrolysis. The gaseous oxygen and hydrogen thereby formed are supplied to at least one oxygen and hydrogen storage units (2a, 2b) which are fluidly connected to the water splitting unit (7) where the gases (i.e. oxygen and hydrogen) are stored. The provision of at least one water splitting unit (7) prevents starving of oxygen and hydrogen in the at least one oxygen and hydrogen storage units (2a, 2b), and thereby ensures continuous availability of gaseous oxygen and hydrogen for the fuel cell (1). The provision of water splitting unit (7) in fluid communication with the water reservoir (3) also reduces the idle time of the system (100), making the system efficient and reliable.

In an alternate embodiment of the disclosure, the oxygen and hydrogen storage units (2a, 2b) are configured such that they can absorb air from atmosphere and supply gaseous oxygen and hydrogen present in atmospheric air into the at least one fuel cell (1). The oxygen and hydrogen storage units (2a, 2b) have separate inlets which are exposed to atmosphere for the purpose of drawing atmospheric air into them. Gaseous oxygen and hydrogen are extracted from the drawn air which are then supplied to the fuel cell (1) to carry out hydrogen-oxygen combination process, leading to the formation of water, accompanied by the release of energy.

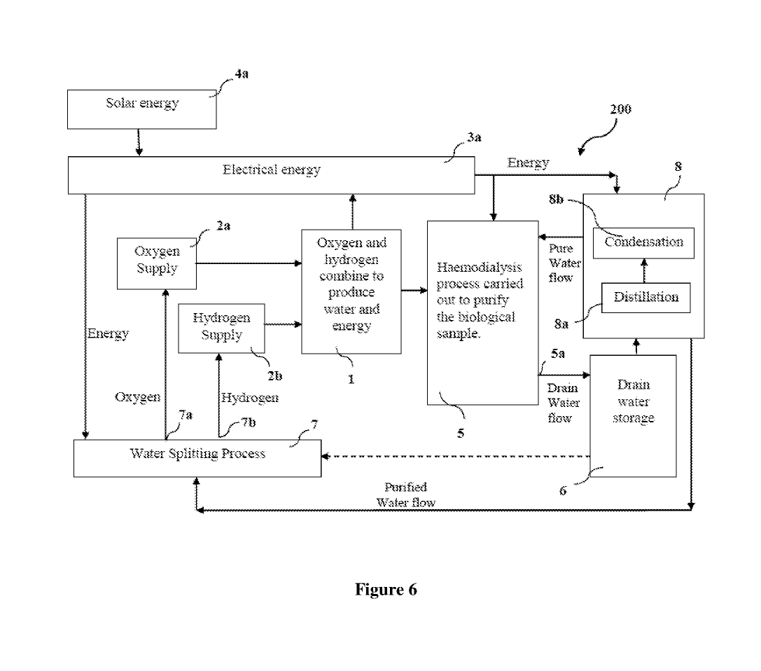

FIG. 5 is another exemplary embodiment of the present disclosure which illustrates the regenerative haemodialysis system (200) of FIG. 4 provided with at least one water purification unit (8). The drain water flowing out of the haemodialyser (5) through water outlet (5a) is subjected to purification in the water purification unit (8). In an embodiment of the disclosure, the water purification unit (8) is provisioned in fluid communication with the water reservoir (6) and is configured to remove impurities present in drain water. In an embodiment of the present disclosure, a part of drain water collected in the water reservoir (6) is routed into the water purification unit (8) to perform purification process, and remaining water is routed directly into the water splitting unit (7) for decomposing water into gaseous oxygen and hydrogen. In an alternate embodiment of the present disclosure, entire volume of drain water that is collected in the water reservoir (6) is routed to the water purification unit (8) where it is purified. The pure water is further subjected to splitting process by chemical decomposition in the at least one water splitting unit (7), thereby producing gaseous oxygen and hydrogen. The water purification unit (8) separates impurities (both solid phase and fluid phase) from water and thereby increases the purity of gaseous hydrogen and oxygen generated in the water splitting unit (7). The energy required to operate the at least one water purification unit (8) is supplied by the at least one energy reservoir (3). Further, the at least one water purification unit (8) is configured to supply purified water into the water compartment of the haemodialysis machine (5) which ensures availability of water to the haemodialysis machine (3) to perform haemodialysis treatment. This is particularly useful in the events such as but not limiting to non-stoichiometric chemical reaction between hydrogen and oxygen gases in the fuel cell (1), and when water produced inside the fuel cell (1) is not sufficient to operate the haemodialysis machine (5). In other words, the water purification unit (8) functions as a standby unit which ensures availability of pure water for supplying into the haemodialysis machine (5).

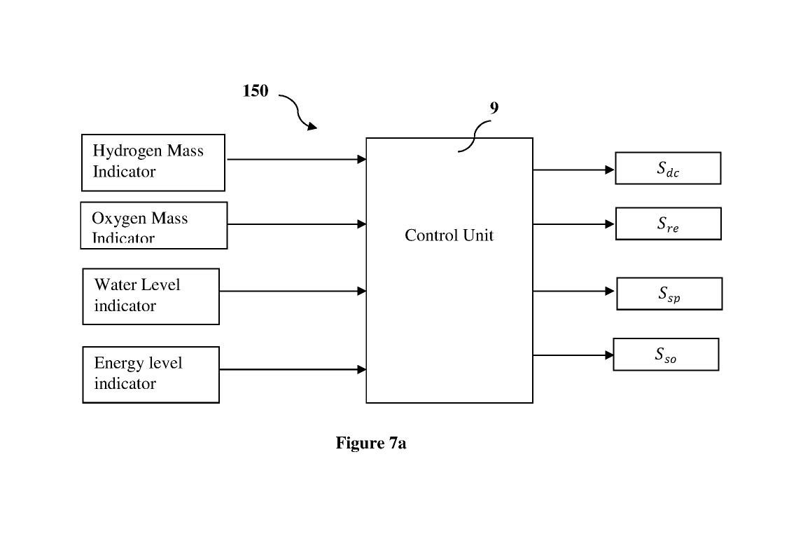

FIG. 6 is another exemplary embodiment of the present disclosure which illustrates a regenerative haemodialysis system (200). The regenerative haemodialysis system (200) as shown in FIG. 6 comprises one or more electric batteries (3a) as energy reservoirs (3), at least one distillation unit (8a) and at least one condensation unit (8b) as water purification unit (8), and at least one solar cell module (4a) as alternate energy source (4). The batteries (3a) used as energy reservoirs (3) are employed to receive and store electrical energy generated in the fuel cell (1), and one or more solar cell modules (4a) include, but are not limited to photovoltaic cells, photoemissive cells and photoconductive cells are used as alternate energy sources (4) to restore battery (3a) charge levels. The one or more batteries (3) therefore get charged whenever there is availability of solar power (i.e. sunlight), thereby reducing the load on the fuel cell (1). The principle of working of solar cell module (4a) (i.e. photoelectric devices), is well known in the art, and hence the same is not discussed in detail for the purpose of simplicity. However, when there is no availability of solar radiation for photoelectric effect to take place, the one or more batteries (3a) get charged by electricity generated in the fuel cell (1). The solar cell module (4a) explained above is only an example to an alternate energy source (4) which can be used to keep the one or more batteries (3a) in charged state, and must not be construed as the only power source used to increase the energy levels in the at least one battery (3) as the person skilled in the art would employ other types of alternate energy sources without departing from scope of present disclosure.

Further, as shown in FIG. 6, the at least one water purification unit (8) comprises at least one distillation unit (8a) and at least one condensation unit (8b). The distillation unit (8a) is fluidly connected to the at least water reservoir (3) and is configured to separate fluidic impurities present in the drain water. The basic principle of purification by distillation, as is known in the art, is separation of various fluids present in a mixture of fluids by virtue of difference in volatilities. The lighter fractions accumulate at the top of the distillation unit (8a) and heavier fractions get accumulated at relatively lower positions of the distillation unit (8a). The fractions are removed from different heights of the distillation unit (8a) and are reclaimed by processes, include, but are not limited to condensation. Accordingly, in the present disclosure, the drain water collected in the water reservoir (6) is subjected to distillation in the distillation unit (8a) where the water is heated to saturated state, so that water evaporates to form saturated steam leaving behind the unwanted fluidic and solid impurities. The unwanted fluidic and solid impurities having more density than water get collected at lower portions of the distillation unit (8a). The vaporized water rises to top of the distillation unit (8a) which is then routed into a condensation unit (8b). In condensation unit (8b), pure water in vaporized state loses latent heat and condenses into liquid. The pure water so formed by condensation is supplied to haemodialysis machine (5) for carrying out the haemodialysis treatment. Alternatively, the pure water obtained can be supplied to the water splitting unit (7) for regenerating the gaseous hydrogen and oxygen.

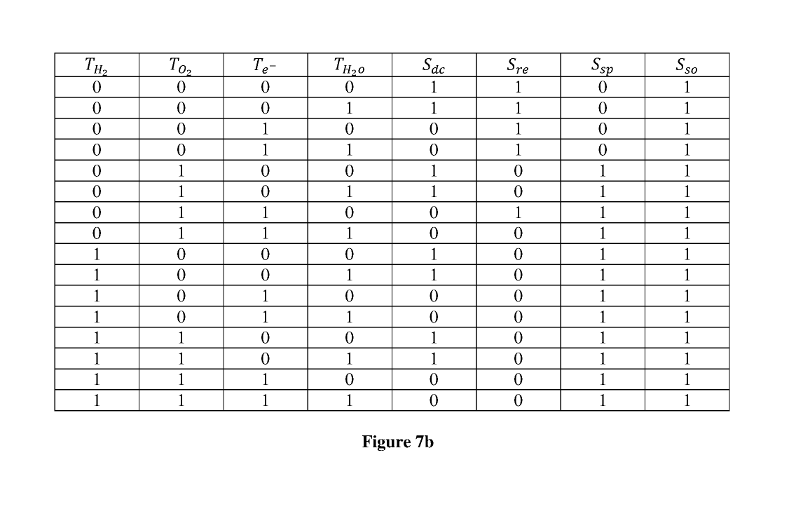

Now referring to FIG. 7a which is an exemplary embodiment of the present disclosure, a control system (150) associated with the hemodialysis system (200) of FIGS. 3-6 is illustrated. The control unit (150) is provisioned in the haemodialysis system (200) to control operations of associated components for balanced, self-sustained supply of input ingredients (oxygen, hydrogen, water and electricity) to the haemodialysis machine (5). The control system (150) is provided with a control unit (9) which may be deployed external to the haemodialysis system (200) or may be integrated within (i.e. internally) the system (200). Further, one or more sensors (not shown) are provided in the haemodialysis system (200), and are interfaced with the control unit (9) for detecting and indicating the energy level in the at least one energy reservoir (3). The control unit (9) is also configured with controller logic to suitably regulate the flow rates of input ingredients required to operate the haemodialysis system (200) being oxygen, hydrogen, water and electricity. The control system (150) is further configured with a quantity indicator system which senses the preset quantity of input ingredients. Accordingly, the control unit (150) generates appropriate signals based on decrease in level or increase in level of the input ingredients with reference to a threshold level. This correspondingly decides whether to increase the rate of generation of input ingredients in their respective chambers or to increase the rate at which they are consumed, so that a balance in levels of these ingredients is maintained. In other words, the ingredients are restored as soon as they are generated and vice versa. This makes the haemodialysis system (200) and its components to operate uninterruptedly, and for a long duration of time. In an embodiment of the present disclosure, the control unit (9) includes but not limiting to electronic controller such as but not limiting to microcontrollers, microprocessor based control units, or hydraulic controllers.

FIG. 7b illustrates an exemplary mode of operation of control system (150) in the haemodialysis system (200) for regulating flow of input ingredients. The sequence of operations performed by the control system (150) is as follows: Based on the input indicator signal, the control unit (9) generates a signal either to “ON” condition or to “OFF” condition designated by S_{dc}, S_{rc}, Sₛₚ and Sₛₒ, each of which acts as switching signal either to initiate a process, or to end a processes. The process includes distillation followed by condensation to purify drain water, hydrogen-oxygen recombining process to create water, water splitting process to regenerate gaseous hydrogen and oxygen, and conversion of alternate energy generated in the alternate energy source into convenient energy form which is stored in the energy reservoir. One of the specific conditions where energy reservoir being used is a battery and energy stored in the energy reservoir is in the form of electrical energy, is shown below. However, the below mentioned condition should not be construed as the only condition of the above mentioned logic and is not in any way limiting the scope of the present disclosure.

The various switching conditions based on the input signal are mapped on to a truth table shown in FIG. 7b, and the truth table is stored in a memory unit [not shown] associated with the control unit (9) in the form of look-up table. The controller (9) refers to the pre-set values stored in the loo-up table for regulating various input ingredients during operation of the haemodialysis system (200).

The various conditions stored in the look-up table include:
1. Switching ON condition signal as S_{dc}=1 is derived when the battery is charged above the threshold level.
2. Switching OFF condition signal as S_{dc}=0 is derived when the battery is charged below the threshold level
3. Switching ON condition signal as Sᵣₑ=1 is derived when the battery charge condition is below the set level for the electrical charge capacity.
4. Switching OFF condition signal as Sᵣₑ=0 is derived when the battery charge condition is below the set level for the electrical charge capacity.
5. Switching ON condition as Sₛₚ=1 is derived when Hydrogen or oxygen is below the threshold quantity.
6. Switching OFF condition as Sₛₚ=0 is derived when Hydrogen or oxygen is below the threshold quantity.
7. Switching ON condition as Sₛₒ=1 is derived whenever the alternate energy is available, but in the current stated case logic condition it is always set as ON condition.

In an embodiment of the present disclosure, the regenerative haemodialysis system (200) is made portable so that the system can be transported to patients' vicinity where the dialysis treatment is carried out. The necessary conveying/transporting arrangement which makes the system portable is powered by the energy reservoir (3).

In an embodiment of the present disclosure, there is provided a bus-propulsion system which is used to transport the regenerative haemodialysis system (200) from one place to another, and thereby makes the system portable. The bus propulsion system is configured to use energy from the at least one energy reservoir (3) as a part of energy required for operation.

In an embodiment of the present disclosure, there is provided a method for operating a portable regenerative haemodialysis system (200). The method comprises steps of firstly generating water and energy in the at least one fuel cell (1) by the electro-chemical reaction between gaseous oxygen and hydrogen taking place in the fuel cell (1). The gaseous oxygen and hydrogen are supplied to the fuel cell (1) by at least one oxygen and hydrogen storage units (2a, 2b). In an embodiment of the present disclosure, the flow of oxygen and hydrogen from the at least one oxygen and hydrogen storage units (2a, 2b) to the at least one fuel cell (1) are regulated by at one flow regulation unit provisioned in flow passages in between the oxygen and hydrogen storage units (2a, 2b) and the fuel cell (1). The gaseous hydrogen and oxygen from storage units (2a, 2b) flow into the fuel cell (1) and dissociate at their respective electrodes to form water as end-product, producing electro motive force across the electrodes along with some quantity of heat. The energy produced in the fuel cell (1) is stored in at least one energy reservoir (3). Further, the method comprises of supplying water generated in the fuel cell (1) to the haemodialyser (5) to perform biological sample purification. At the same time, the energy stored in the at least one energy reservoir (3) is supplied to the haemodialyser (5) to carry out purification process. After carrying out the haemodialysis process, the waste water is expelled out of the haemodialyser (5) and is regenerated to make it reusable. The regeneration of water is carried out in a regenerative system and is carried out in the following steps: purifying the drain water collected in the at least one water reservoir (6) by at least one water purification unit (8) to generate water with high degree of purity. In an embodiment of the present disclosure, the method comprises act of routing at least a part of purified water into an at least one water splitting unit (7) to regenerate oxygen and hydrogen by chemical decomposition. The gaseous oxygen and hydrogen so regenerated are further routed to the oxygen and hydrogen storage units (2a, 2b) for supplying them to the fuel cell (1) to produce water and energy, thereby facilitating next cycle of haemodialysis process. The necessary energy required to operate the water splitting unit (7), water purification unit (8) and the haemodialysis machine (5) is supplied by the energy reservoir (3). In an embodiment of the present disclosure, the method also comprises act of supplying energy from at least one alternate energy source (4) to the energy reservoir (3).

Advantage(s):

The present disclosure provides a regenerative portable haemodialysis system in which input ingredients such as water and electricity are regenerated, resulting in considerable savings in water and electricity consumptions.

The present disclosure provides a regenerative portable haemodialysis system in which a chemical reaction (combination) between molecular hydrogen and molecular oxygen in a hydrogen-oxygen fuel cell produces water and electricity as end products. The water so produced will have high degree of purity which cannot be achieved by conventional purification methods. Also, the system eliminates the need of separate source of electricity to operate the haemodialysis system, as electricity is generated with water within the system itself. This makes the system compact and economical.

The present disclosure provides a haemodialysis system which can operate uninterruptedly due to continuous availability of water and electricity. This makes the haemodialysis system reliable and feasible to carry out haemodialysis treatment in places where conventional haemodialysis treatments are not viable. This helps the system portable, and accessibility of the system to remote areas can be achieved.

Equivalents:

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Table of referral numerals:

| Reference Number | Description |
|---|---|
| 100 | Haemodialysis system |
| 1 | Fuel cell |
| 2a | Oxygen storage unit |
| 2b | Hydrogen storage unit |
| 3 | Energy reservoir |
| 3a | Battery |
| 4 | Alternate energy source |
| 4a | Solar cell module |
| 5 | Hemodialysis machine/haemodialyser |
| 6 | Water reservoir |
| 7 | Water splitting unit |
| 7a, 7b | Outlet ports of water splitting unit |
| 8 | Water purification unit |
| 8a | Distillation unit |
| 8b | Condensation unit |
| 200 | Regenerative haemodialysis system |
| 150 | Control system |
| 9 | Control unit |

1. A portable haemodialysis system, comprising:
at least one haemodialyser configured to purify a biological sample;
at least one fuel cell fluidly connected to the at least one haemodialyser, the at least one fuel cell is adapted to receive oxygen and hydrogen from at least one oxygen and hydrogen storage units to generate energy and water; and
at least one energy reservoir connected to the at least one fuel cell, the at least one energy reservoir is configured to store the energy generated in the at least one fuel cell;
wherein, the water generated in the at least one fuel cell is supplied to the at least one haemodialyser for purification of the biological sample, and the energy stored in the at least one energy reservoir is used to power the haemodialyser during operation.

2. The system as claimed in claim 1, wherein the biological sample is a corporeal fluid.

3. The system as claimed in claim 1, wherein the at least one fuel cell is a Hydrogen-Oxygen fuel cell.

4. The system as claimed in claim 1, wherein the at least one energy reservoir is a battery.

5. The system as claimed in claim 1, wherein the at least one energy reservoir is connected to at least one alternate energy source.

6. The system as claimed in claim 1, includes at least one water reservoir fluidly connected to a water outlet of the haemodialyser.

7. The system as claimed in claim 1, includes at least one water purification unit fluidly connected to the water reservoir and the haemodialyser.

8. The system as claimed in claim 7, wherein the at least one water purification unit is configured to purify the water used for purification of the biological sample.

9. The system as claimed in claim 1, includes at least one water splitting unit fluidly connected to at least one of the at least one water purification unit and the at least one water reservoir.

10. The system as claimed in claim 9, wherein the at least one water splitting unit is configured to regenerate gaseous hydrogen and oxygen by decomposing the pure water.

11. The system as claimed in claim 1, wherein the at least one water splitting unit comprises outlet ports, the outlet ports are fluidly connected to the oxygen and hydrogen storage units for supplying gaseous oxygen and hydrogen.

12. The system as claimed in claim 1, includes at least one sensor interfaced with a control unit for detecting and indicating the energy level in the at least one energy reservoir.

13. The system as claimed in claim 1, includes at least one flow regulation unit provisioned in flow passages between the oxygen and hydrogen storage units and the fuel cell.

14. The system as claimed in claim 13, wherein the at least one flow regulation unit is interfaced with the control unit for regulating the flow of hydrogen and oxygen to the at least one fuel cell.

15. A regenerative portable haemodialysis system, comprising:
at least one haemodialyser configured to purify a biological sample;
at least one fuel cell fluidly connected to the at least one haemodialyser, the at least one fuel cell is adapted to receive oxygen and hydrogen from at least one oxygen and hydrogen storage units, and is configured to generate energy and water;
at least one energy reservoir connected to the at least one fuel cell, the at least one energy reservoir is configured to store energy generated in the at least one fuel cell;
wherein, the water generated in the at least one fuel cell is supplied to the at least one haemodialyser for purification of the biological sample, and the energy stored in the at least one energy reservoir is used to power the haemodialyser during operation; and
a regenerative system for regenerating water and energy used for haemodialysis, the regenerative system comprising:
at least one water purification unit fluidly connected to water outlet of the haemodialyser, the at least one water purification unit is configured to purify the water used for purification of the biological sample; and
at least one water splitting unit fluidly connected to the at least one water purification unit, the at least one water splitting unit is configured to regenerate gaseous hydrogen and oxygen by decomposing the water, and supply the gaseous oxygen and hydrogen to the at least one oxygen and hydrogen storage units.

16. The system as claimed in claim 15, wherein the at least one energy reservoir is configured to power the regenerative system for regenerating water and energy.

17. The system as claimed in claim 15, wherein the at least one energy reservoir is connected to at least one alternate energy source.

18. The system as claimed in claim 15, includes at least one water reservoir fluidly connected to water outlet of the haemodialyser.

19. A method for operating a regenerative portable haemodialysis system, the method comprising acts of:
generating water and energy using at least one fuel cell by supplying oxygen and hydrogen, wherein the at least one fuel cell is configured to receive oxygen and hydrogen from at least one oxygen and hydrogen storage tanks;
storing the energy generated by the at least one fuel cell in at least one energy reservoir;
supplying the water generated by the at least one fuel cell to the at least one haemodialyser for purification of the biological sample, and supplying the energy stored in the at least one energy reservoir to power the haemodialyser during operation; and
regenerating water and energy used for haemodialysis by a regenerative system, the regeneration comprising steps of:
purifying the water in at least one water purification unit, wherein the at least one water purification unit receives water from water outlet of the haemodialyser;
regenerating gaseous oxygen and hydrogen by decomposing the water in at least one water splitting unit, wherein the at least one water splitting unit receives water from the at least one water purification unit; and
supplying regenerated oxygen and hydrogen to the at least one oxygen and hydrogen storage units, wherein the at least one oxygen and hydrogen storage units are configured to receive oxygen and hydrogen from the at least one water splitting unit.

20. The method as claimed in claim 19 comprises an act of supplying energy from at least one alternate energy source to the at least one energy reservoir.

21. The method as claimed in claim 19 comprises an act of regulating flow of oxygen and hydrogen from the at least one oxygen and hydrogen storage units to the at least one fuel cell by at least one flow regulation unit.
